(19) United States
(12) Patent Application Publication
(10) Pub. No.: US 2008/0190789 A1
(43) Pub. Date: Aug. 14, 2008

(54) Air Freshener Package

(75) Inventors: Daniel D'Amico, South Salem, NY (US); Jeremy R. Brookes, Lexington, KY (US); David Sissom, Hollywood, FL (US)

Correspondence Address: David W Carrithers;Carrithers Law Office, One Paragon Centre, 6060 Dutchman's Lane, Suite 140, Louisville, KY, 40205, US

(73) Assignee: ASHLAND LICENSING AND INTELLECTUAL PROPERTY, LLC., Dublin, OH (US)

(21) Appl. No.: 10/562,570

(22) PCT Filed: Dec. 1, 2005
(86) PCT No.: PCT/US2005/043448

§ 371 Date: Feb. 15, 2008

Related U.S. Application Data
(60) Provisional application No. 60/632,037, filed on Dec. 1, 2004.

(51) Int. Cl. B65D 69/00; A61L 9/12; A61L 9/00; B65D 73/00

(52) U.S. Cl. 206/229; 422/123; 206/485; 239/34

(57) ABSTRACT

An air freshener package for containing an air freshener and a fragrance emitting substance. The blister type package has one compartment for the air freshener unit and another sample compartment forming a bubble or blister for a separate sample of the scent emitting material. The air freshener unit and sample compartment have a corresponding fragrance. The sample compartment forms a pressure responsive selected area on the package and is designed of a rupturable ventable material. The sample compartment can be deformed by finger pressure decreasing the volume of the compartment and thereby causing a puff of scented air to exhaust to atmosphere through a discharger opening. In an alternate embodiment, the finger pressure is sufficient to rupture a fragrance containing bead filled with fragrance air or liquid which emits a fragrance which is released through a vent or a pin hole formed in the second sample compartment to the environment. The user can sample the product while the integrity and seal of the air freshener unit and fragrance substance therein remains intact and fresh.

This application claims priority from U.S. Provisional Application Ser. No. 60/632,037 filed on Dec. 1, 2005 the entirety of which is incorporated herein.

### FIELD OF INVENTION

This invention relates generally to air freshener units that contain a fragrance emitting substance and more particularly to a package for an air freshener unit and to a packaged air freshener unit wherein the package has one compartment for the air freshener unit and another sample compartment forming a bubble or blister for a separate sample of the scent emitting material. The air freshener unit and sample compartment have a corresponding fragrance. The sample compartment forms a pressure responsive selected area on the package and be designed of a rupturable material. The bubble can be deformed by finger pressure decreasing the volume of the compartment and thereby causing a puff of scented air to exhaust to atmosphere through a discharger opening. In an alternate embodiment, the finger pressure is sufficient to rupture a fragrance containing bead filled with fragrance air or liquid which emits a fragrance which is released through a vent or a pin hole formed in the second sample compartment. Since the sample and the air freshener produce the same scent a purchaser of the packaged unit has no later surprises or disappointments. The user can sample the product while the integrity and seal of the air freshener unit and fragrance substance therein remains intact and fresh.

### BACKGROUND OF THE INVENTION

### Description of the Prior Art

There are numerous, what maybe considered personal, scent emitting devices of which many are intended primarily for use in automobiles, homes, and public rest rooms. Slow release scent emitters are known in which a scent emitting substance is absorbed in a polymeric material for example, polythene, ethyl vinyl acetate or ethyl vinyl alcohol or a cellulosic material. By way of example reference maybe had to U.S. Pat. No. 2,169,055 by Overshiner et al which discloses a slow release scent emitter using a cellulosic compound.

The scent emitter maybe variously formed, shaped and/or in various sizes and formulated and designed for consistency in the release of a scent over a long period of time and in this regard a long period of time is considered by applicant to be up to at least 45 days. The particular form of the air freshener unit and/or form, shape or size of the scent emitting material is not an essential part of the present invention other than having the scent emitter physically in a solid state and most preferably in the form of beads which maybe either uniform or non-uniform in size and preferably with the scent release being consistent over a long period of time. Or course, any fragrant emitting substrate could be utilized in the air freshener package of the present invention.

In buying existing air freshener units one is uncertain as to what scent what scent will be emitted therefrom until they open the package to use the same in the cabin of their vehicle. This can result in a lot of disappointments or surprises because of the scent differing from that which was expected. In marketing air freshener devices a choice has had to be made between having a long shelf life for the unit by keeping the scent emitter sealed until opened for use or having them emit a scent for customers to sniff before purchasing. The latter is not practical because one purchasing the unit would have no idea or way of knowing as to how much life remained in the unit.

A squeezable scent dispensing automobile are freshener unit is disclosed in U.S. Pat. No. 6,254,836 granted Jul. 6, 2001 to D. Fry. The purpose of squeezing the unit is to discharge the scent into the cabin of the motor vehicle; however, none of the conventional air freshener packages utilize an air freshener compartment and separate corresponding sample compartment forming a blister or bubble containing a bead or particle of the fragrance vapor, liquid, semi-solid, or solid material including means to disperse same to the atmosphere as a fragrance sample.

### SUMMARY OF INVENTION

The present invention provides a package for an air freshener unit wherein a customer can, by finger pressure, cause release of a puff of scented air to atmosphere from an independent separate sample captively retained in one part of the package and wherein such scent corresponds to the scent that will be emitted from an air freshener unit which is retained in another part of the package.

The air freshener invention includes a fragrance emitting substance contained within an air freshener enclosed within a package wherein the package has one compartment for the air freshener unit and another sample compartment forming a bubble or blister for a separate sample of the scent emitting material. The air freshener unit and sample compartment have a corresponding fragrance. The sample compartment forms a pressure responsive selected area on the package and may have be designed of a rupturable material. The bubble can be deformed by finger pressure decreasing the volume of the compartment and thereby causing a puff of scented air to exhaust to atmosphere through a discharger opening. In another preferred embodiment, the second sample compartment includes a vent such as a pin hole therein so that finger pressure is sufficient to rupture a fragrance containing bead or film bubble filled with fragrance air or liquid which emits a fragrance.

A further object of the present invention is to provide a packaged air freshener unit wherein a customer can, by finger pressure, cause release of a puff of scented air to atmosphere from an independent separate sample captively retained in one part of the package and wherein such scent corresponds to the scent emitted from an air freshener unit retained in another part of the package.

Another object of the present invention is to provide a packaged air freshener unit as defined in the foregoing wherein the scent emitter comprises a sample compartment in the form of hollow sample compartments selected from beads, sacks, pellets, envelopes, membranes, straws, or other hollow container means filled with a liquid, vapor, semi-solid, granular, powder, or solid fragrance embedded substrate that provides consistency in the release of the scent over a long period of time.

In keeping with the foregoing there is provided in accordance with the present invention a package for an air freshener unit containing a scent emitter substance and intended primarily for use in the cabin of motorized vehicles, said package comprising respective front and rear panels shaped to provide there between first and second spaced apart compartments for captively retaining therein respectively an air freshener unit having material that produces a preselected scent and a sample of such material, said package having an outlet opening communicating with said second compartment and discharging to atmosphere and a finger pressure responsive selected area on said package selectively to vary the volume of said second compartment thereby enabling discharging therefrom a puff of scented air upon applying finger pressure to said selected area on the package.

These an other objects and features of the invention will become apparent to those skilled in the art from the following detailed description and appended claims.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 is an exploded oblique view of an air fresher unit and package therefor provided in accordance with the present invention;

FIG. 2 is a front face view of the air freshener package showing an embodiment of an air freshener in broken line;

FIG. 3 is a sectional view of the air freshener package of FIG. 1 taken along line 3-3 of FIG. 3;

FIG. 4 is an oblique exploded view of an alternate embodiment of an air freshener package showing a first product compartment integrally connecting to a second spaced apart sample compartment;

FIG. 5 is a sectional view taken along line 5-5 of FIG. 4;

FIG. 6 is a sectional view taken along line 6-6 of FIG. 4;

FIG. 7 is a perspective view showing an alternate embodiment of the air freshener package wherein the first product compartment is spaced apart from the second sample compartment;

FIG. 8 is an exploded view of the air freshener package of FIG. 7 wherein the first product compartment is spaced apart from the second sample compartment;

FIG. 9 is a front perspective view of the air freshener package of FIG. 8 containing an air freshener and sample product;

FIG. 10 is a rear perspective view of the air freshener package of FIG. 8 containing an air freshener and sample;

FIG. 11 is a left side perspective view of the air freshener package of FIG. 8 containing an air freshener and sample product;

FIG. 12 is a right side perspective view of the air freshener package of FIG. 8 containing an air freshener and sample product;

FIG. 13 is a top perspective view of the air freshener package of FIG. 8 containing an air freshener and sample product;

FIG. 14 is a bottom perspective view of the air freshener package of FIG. 8 containing an air freshener and sample product;

FIG. 15 is an exploded view of the air freshener device and the scented product contained therein; and

FIG. 16 is an exploded view showing the air freshener device, scented product contained therein, and air freshener package containing the air freshener device or product together with the sample compartment and sample bead which when ruptured released a scent from the sample compartment to the atmosphere.

### DESCRIPTION OF PREFERRED EMBODIMENT

Illustrated in the drawings is an air freshener package 10 comprising a blister type package for air freshener unit 40 having a fragrance emitting substrate therein, is captively retained in a first product compartment 11. The package maybe referred to as a blister package comprising respective front and rear panels 12 and 13 in which at least one of them has a cavity provided therein so that when the two panels are laid one over the other a compartment is provided to receive and captively retain therein an article. In the instant case each panel has a cavity with front panel 12 having a cavity 14 and the rear panel having a cavity 15. Together these cavities provide the aforementioned first compartment 11 that retains therein the air freshener unit 40.

The front and rear panels maybe variously shaped and joined together in any convenient manner such as by thermal welding, adhesives, locking flanges, tongue and groove arrangement, shrink wrap, or molding.

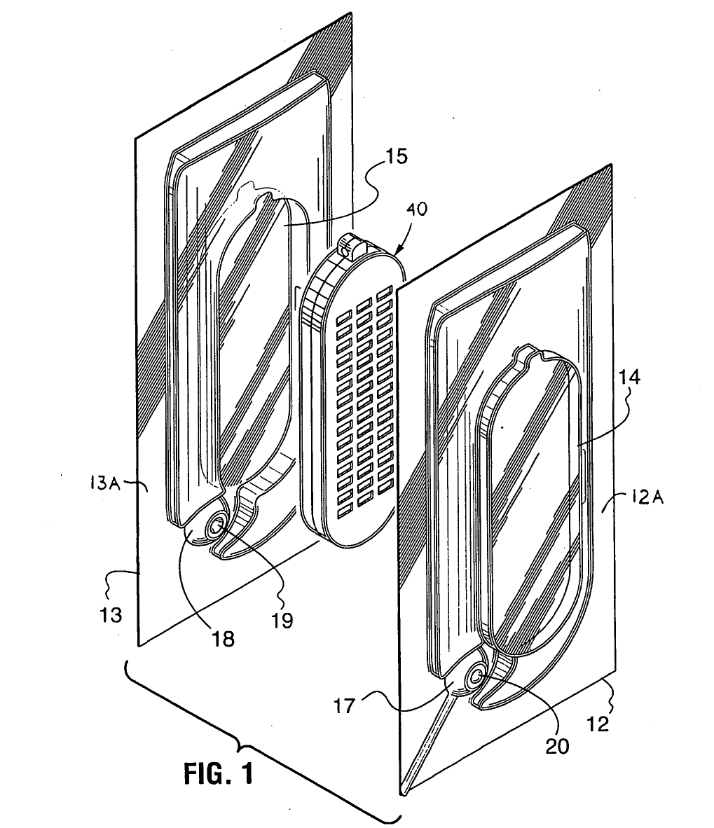

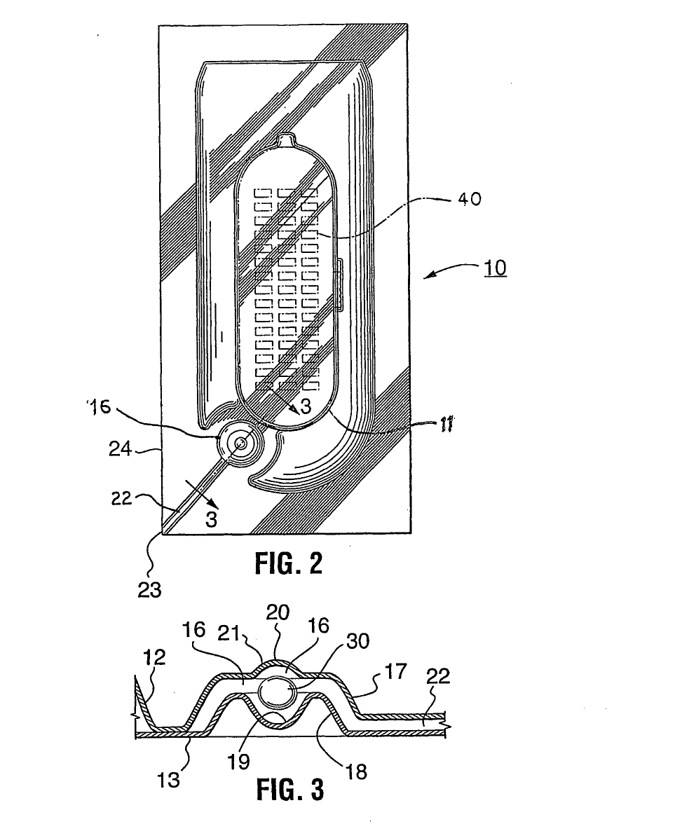

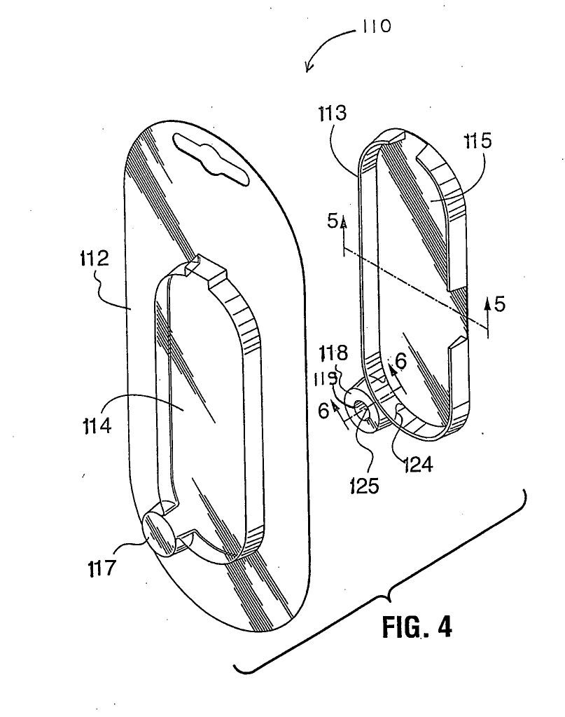

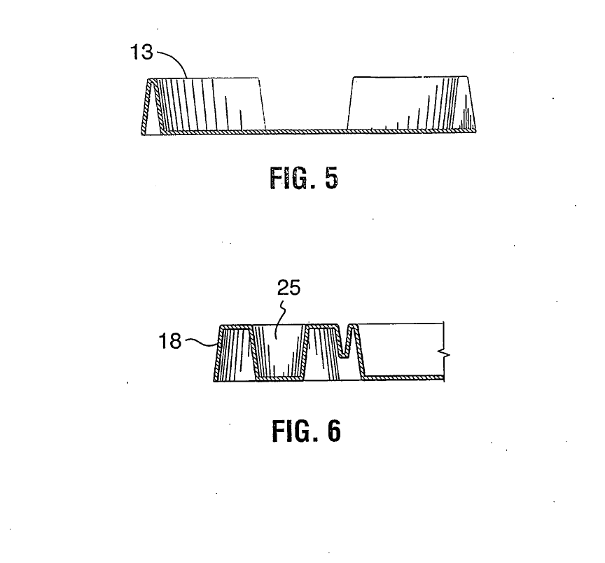

As shown in one preferred embodiment in FIGS. 1-3, the front and rear panels have respective planar peripheral portions 12A, 13A that abut in face to face relation. In the embodiment illustrated in FIGS. 4-6 front panel 12 has a V-shaped channel 12B along a major portion of the periphery and receives therein a flange 13B that projects from the rear panel 13.

In accordance with the present invention the air freshener package 10 has a second sample compartment 16 in the package that captively retains therein a sample 30, (see FIG. 3 ) of the scent emitting material that is the same as that in the air fresher unit 40. It is preferred that the scent emitting material be in the form of beads, for example polyethylene or the like, with one or more beads being retained in the compartment 16. One preferred embodiment contains a single fragrance impregnated bead 30.

The second sample compartment 16 is so constructed and located so as to be readily squeezed or depressed and though suitable outlet means in communication with the compartment exhaust to atmosphere a puff of scented air that can be sniffed. A purchaser of the device thus will know exactly what scent will be emitted from the air freshener unit in the package without any affect on the air freshener unit itself to prevent oxidation of the fragrance or dilution and dispersion of the strength of the scent. The compartments 11, 16 are spaced apart and independent of one another to maintain integrity of the first “product” compartment containing the air freshener and fragrance substrate 60. The second sample compartment 16 is provided by inter-nested truncated conical projections 17, 18 on respective front and rear panels 12, 13. In the embodiment illustrated in FIGS. 1-3, there is concave depression 19 in the projection 18 that lies opposite a convex projection 20 in the top end of the projection 17. The depression 19 and projection 20 are effectively oppositely directed sub-cavities and together provide a bulbous area 21 that captively retains the sample 30 and can readily be squeezed or depressed to drive a puff of scented air through a passage 22 that discharges to atmosphere through an outlet opening 23 in a peripheral edge 24 of the package. In this embodiment, the second sample compartment 16 is spaced inwardly from the outer peripheral edge of the first product compartment 11 of the air freshener package 10. Of course, the second sample compartment 16 can be composed at least partially of a rupturable material such as a film bubble or the like. Moreover, the sample material or bead can be of hollow construction and filled with a vapor of liquid fragrance substrate.

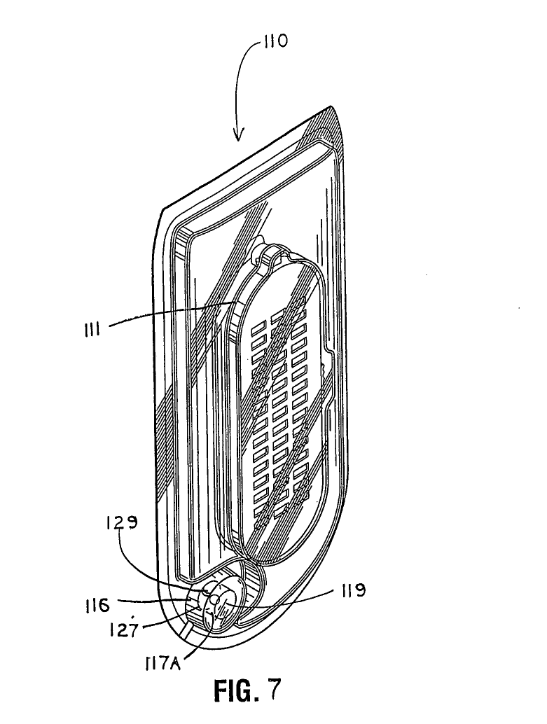

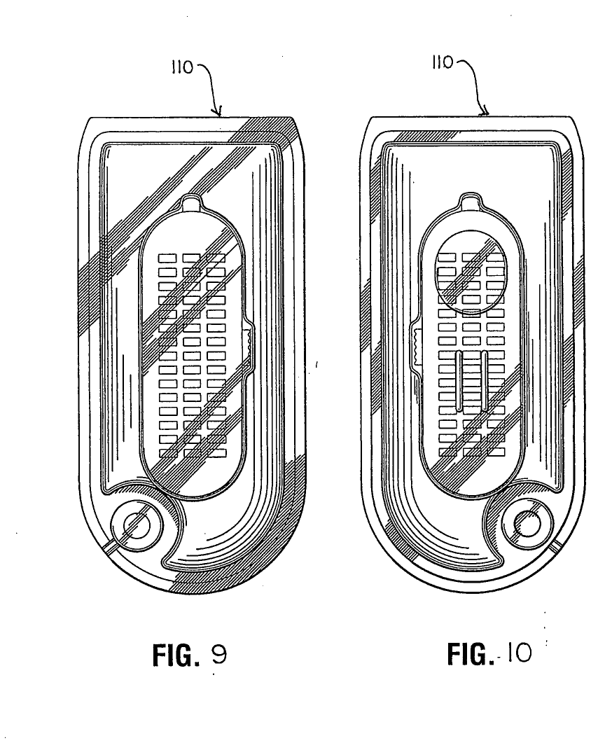

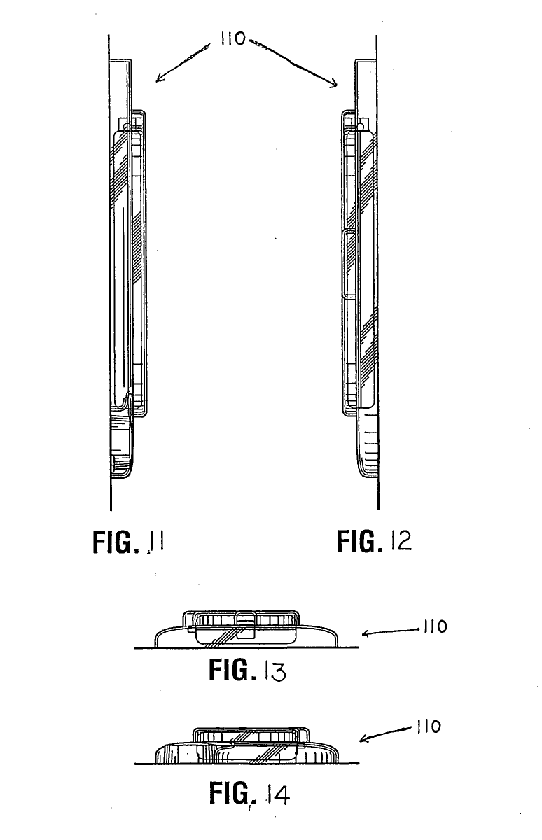

In alternate preferred embodiments illustrated in FIGS. 4-7 and 8-16, the second compartment forms a squeezable second chamber 116 for a sample of scent containing substrate either separately formed or formed integrally attached to the first compartment 111 of the air freshener package 110.

In the preferred embodiment shown in FIGS. 4-7, the second compartment 116 is provided by formations that project outwardly from the peripheral edge 124 of each of the respective front and rear panels 112 and 113. The formations are respective truncated or cylindrical portions 117,118 in the respective front and rear panels 112,113 of the package 110. This outwardly projecting second sample compartment portion maybe referred to as a dog tag assembly which maybe integrally formed with the package panels 112 and 113 respectively, as for example illustrated in the drawings. Also, in this embodiment there is an opening 125 in a wall of the second compartment 116 from which a puff of the scented air can be discharged to the atmosphere. The opening 125 illustrated is provided by crossed slits 129 in a bottom wall of a portion 11 7A that projects into a central depression 119 in the panel member 118. The depression 119 has a bottom wall 127 that, when the panels 112 and 113 are assembled to provide a package 110, is spaced from the wall having the slits therein providing there between space for the bead 30. The slits 129 (one or more) can essentially provide a self closing opening which is opened by deformation of the wall and/or pressure build up in the second compartment upon squeezing the same. In place of the slits one or more holes or rupturable thin or creased portions maybe provided in a wall of the second compartment 116.

In the embodiment illustrated in FIG. 4-7 the truncated conical portions 117, 118 may for example have the side walls thereof contacting one another in which case the end walls of such truncated portions are spaced from one another providing there between the aforementioned second compartment for the sample 30. Alternatively such inter-nested conical portions or cylindrical portions maybe the same or similar to that illustrated in FIG. 3.

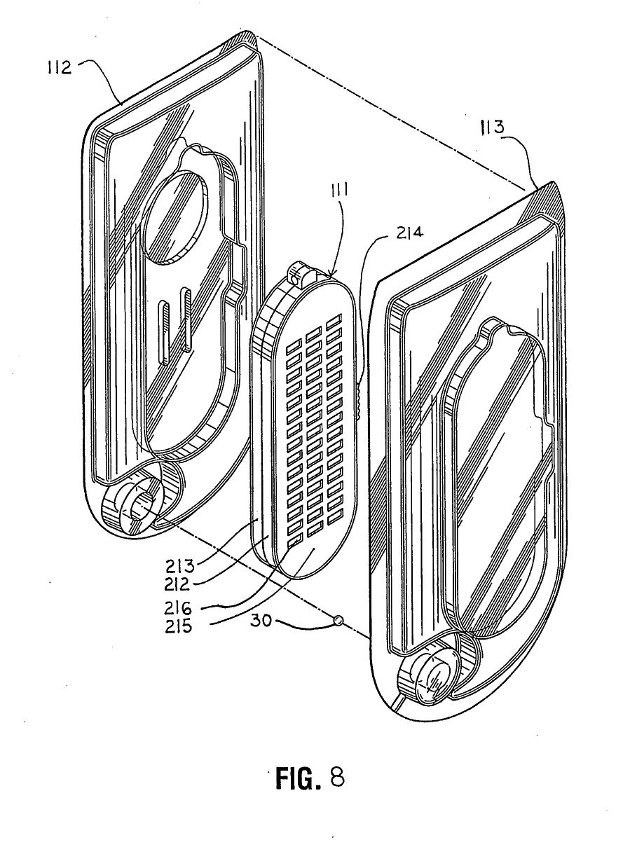

In another preferred embodiment, shown in FIGS. 8-16, the outwardly projecting second sample compartment portion 116 may be formed separate from the package and then attached thereto by suitable means such as an adhesive, snap lock, or friction fit. Also, in this embodiment there is an opening 125 in a wall of the second compartment 116 from which a puff of the scented air can be discharged to the atmosphere. The opening 125 illustrated is provided by crossed slits 129 in a bottom wall of a portion 117A that projects into a central depression 119 in the panel member 118. The depression 119 has a bottom wall 127 that, when the panels 112 and 113 are assembled to provide a package 110, is spaced from the wall having the slits therein providing there between space for the bead 30. The slits 129 (one or more) can essentially provide a self closing opening which is opened by deformation of the wall and/or pressure build up in the second compartment upon squeezing the same. In place of the slits one or more holes maybe provided in a wall of the second compartment 116.

As noted heretofore, the front and rear panels maybe joined together in any known manner for example by staples, tape, adhesive and/or friction fit.

In the embodiment illustrated in Figure, the truncated conical portions 117, 118 may for example have the side walls thereof contacting one another in which case the end walls of such truncated portions are spaced from one another providing there between the aforementioned second compartment for the sample 30. Alternatively such inter-nested conical portions maybe the same or similar to that illustrated in FIG. 3.

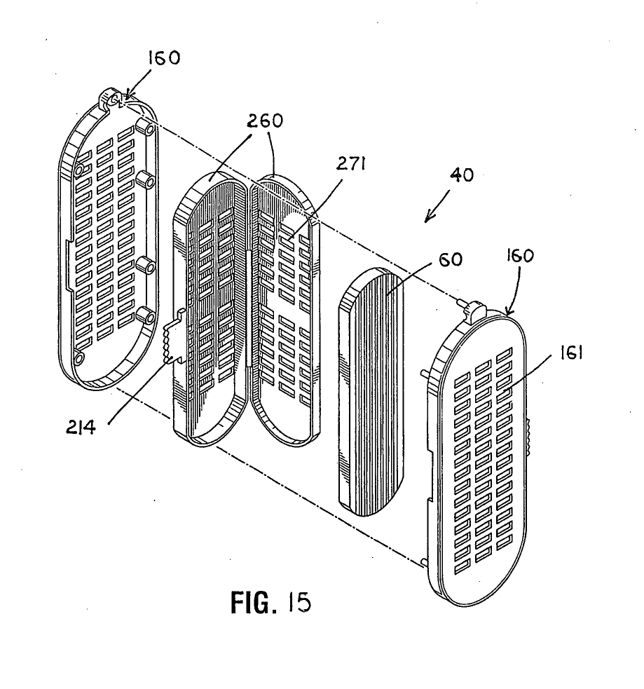

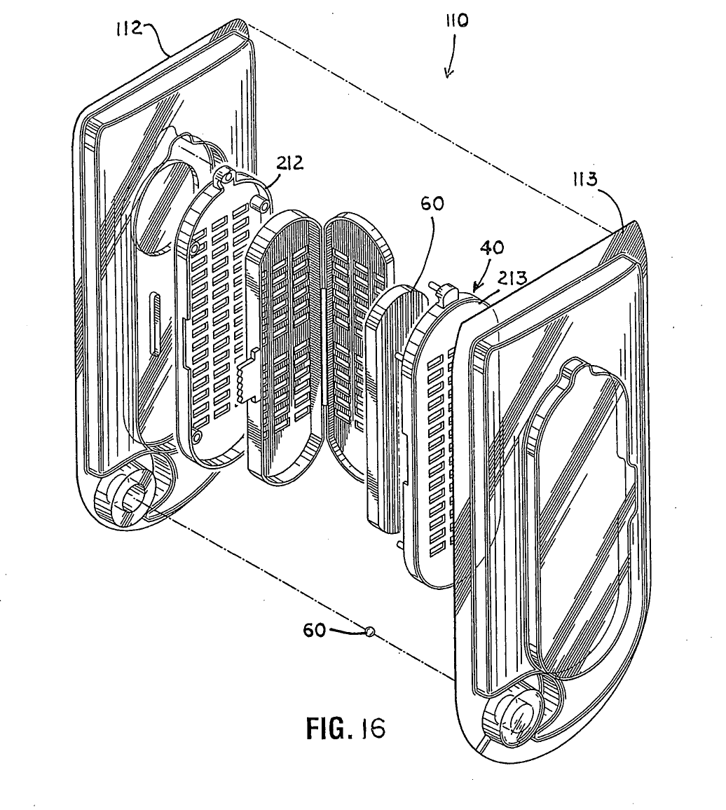

The air freshener 111 may comprise any number of shapes or sizes. One example is best shown in FIGS. 15 and 16 whereby the air freshener comprises a shell 160 formed by a front oblong concave panel 212 and a corresponding rear oblong concave panel 213 attached together forming a cavity or compartment therein between and having openings or louvers 161 formed in the face thereof. An air freshener substrate 60 is held within the cavity.

In another preferred embodiment, a second smaller inner shell or substrate case 260 nested within the shell 160. The substrate case 260 includes openings or louvers 271 which correspond in size and shape to openings 161 in the shell 160. A thumb slide 214 is used to slide louvers up or down to cover slits or other openings 216 formed in the face 215 of at least one of the panels 212, 213.

The term air freshener as used throughout herein is intended to be generic to fragrance producing substances as well as disinfectants and the like that can be transferred to a flowing stream of air so to improve the quality of such air.

The air freshener and the present invention is a full air flow through device consisting generally of a substrate impregnated with a substance that is time released into the surrounding air and in which such substance when in the air can upon appropriate choice provide a pleasant aroma.

A particular scent or aroma can be distributed throughout the ventilation and cabin area and provide a means for aroma therapy with selected scents and fragrances. The fragrance oil can be any natural substance, synthetic material, (incorporating aldehydes, ketones, esters, and other chemical constituents), or combinations thereof which is known in the art and suitable for use in candles for imparting an odor, aroma, or fragrance. Suitable natural and synthetic fragrance/flavor substances include those compiled by the U.S. Food and Drug Administration in Title 21 of the Code of Federal Regulations, Sections 172.510 and 172.515 respectively. Suitable fragrances include spice oil, flower oil, and fruit oil. The fragrance oil may contain fragrance components, for example benzaldehydes, phenols, cinnamic aldehydes and esters, octadienes, dienes, cyclohexadienes, and terpenes.

The fragrances may comprise an essential oil in a carrier such as water and/or alcohol or other organic solvent or even a perfume. The fragrance may be that of a fruit and berry scents such as: citrus, almond, apple, cherry, grape, pear, pineapple, orange, strawberry, raspberry, and musk; flower scents such as lavender, rose, iris, carnation, gardenia, tea rose, violet, hyacinth, magnolia, mimosa, honeysuckle, jasmine, narcissus, orange blossom, orchids, sweet pea, tuberose, and lilac; forest and herbal smells such as evergreen cedar, pine, sassafras, and spruce; essential oils such as spice, peppermint, vanilla, spearmint; and various other fragrances such as leather, new car odor, acacia, cassie, cypre, cyclamen, fern, hawthorn and the like. The fragrance is not critical so long as it is compatible with the polymer impregnated with the time release carrier. The level of fragrance can vary up to about 100 percent by weight and more preferably from 0.001 to about 100 percent by weight. Other agents such as surfactants, emulsifiers, and polymers can be used to encapsulate the fragrance.

The carrier and means of controlling the release of the selected fragrance is dependent upon many variables including time and temperature with respect to the environment inside and outside of the vehicle. The fragrance and substrate composition comprising of 10% to 99.5% by weight of a suitable substrate and from 0.5% to 90% by weight of a fragrance oil.

It is also contemplated that the scented material may be composed of 100 percent of the fragrant raw material without a solvent or essential oil. Furthermore, the fragrance can be imparted to the air freshener absorbent material as an aerosol or gas vapor separate from, or in combination with a liquid substrate.

In one preferred embodiment, the fragrance can be absorbed by a polymeric material such as an ethyl vinyl acetate polymer simply by being disposed in close proximity thereto. Ethyl vinyl alcohol resin is another polymer having good scent absorbent capabilities.

One preferred group of copolymers of ethylene and vinyl acetate which absorb odors are produced by DuPont Industrial Polymers under the trade name of ELVAX resins which generally range in vinyl acetate content from 9 to 40% and have melt indexes from 0.3 to 500 dg/min. Moreover, the ELVAX resin from DuPont does not incorporate a plasticizer which could interfere with the scent absorbing capability of the polymer. The polymer can be formed in any desired shape prior to treatment or under low heat conditions (70° C. or less). The ELVAX resin can be extruded or molded and blended with polyethylene, polypropylene, ABS resins, thermoplastic rubber nitril rubbers, natural rubber and other elastomers. The DUPONT Industrial Polymers ELVAX Grade Selection Guide pages 1-7 is incorporated by reference herein.

The amount of scent adsorbed into the vinyl acetate substrate and the control release mechanism is dependent upon the time and temperature relationship between the scent and substrate as well as the volatility of the scent, content or density of the polymer, level of polymerization, surface area (porosity), and pore size and structure of the polymer substrate which in the instant example is a vinyl acetate absorbent material. Thus, the impregnation occurs through passive adsorption and the time release mechanism occurs through passive diffusion.

As described in U.S. Pat. No. 2,169,055 by Overshiner et al., a cellulose compound such as cellulose acetate or cellulose nitrate can be imparted with a compatible scent imparting material which is released over an extended period of time. The cellulose compound can be produced in a solution with an organic solvent such as acetone and 1,4 diethylene oxide, and adding a scent imparting essential oil to the solution. Plasticizers such as diethyl phthalate and tri-acetic acid ester of glycerin may be used to impart flexibility to the material. The solution may be formed in to sheets, bars, films or the like by casting or molding. The solvents evaporate and a porous cellular structure is formed which releases the essential oil at a rate to provide an odor lasting for several months. Moreover, as shown in FIG. 6, the scent impregnated polymer is over wrapped and crimped at desired intervals to form an air tight seal utilizing a nonpermeable vacuum sealed material to prevent premature release of the scent in storage. Thus, the product can be air evacuated, or even vacuum sealed, in order to prevent release of the scent from the polymeric substrate prior to installation. The strength of the scent may also be determined by the quantity of the individual polymer units.

Micro encapsulation provides a method of controlling the release of scent in liquid form by enclosing the scent within hollow shells of differing size and wall thickness which can be dissolved or ruptured at different intervals to provide a generally steady supply of scent exposed to the environment. Moreover, semipermeable shells which allow escape through the shell wall without shell rupture exist to control the release of a scent and can be incorporated into a bead 30 as shown in the figures.

Another method of controlling the release of the scent from a carrier is to use a homogeneous semipermeable material containing the active ingredient as a pure impregnate, solute or precipitate. The semipermeable material serves as the carrier from which it can only slowly escape by solution, diffusion, evaporation or combinations thereof. The characteristics of the carrier material depends on properties such as pore size, compatibility with the environment, liquid content, temperature of environment, wet-ability, and processing parameters.

One type of polymer liquid composite material prepared which can be utilized in the present invention is an organic or inorganic cellulose ester such as cellulose triacetate or cellulose nitrate as vehicles for the controlled release of active materials into the environment. As described in U.S. Pat. No. 3,985,298 by Nichols and incorporated by reference herein, the composite material can be formed to prepare transparent coherent materials formed as films, fibers or microspheres. The scent may be carried in a fluid which may comprise water, alcohol, ether, aliphatic and aromatic hydrocarbons, ketones, esters, and combinations thereof together with other chemical constituents. For instance, the chemical composition comprising the selected fragrance or scent or other chemical such as a disinfectant or mildew or fungicide may be incorporated as a component in an aerosol propellant, gas, or liquid containing a solvent carrier such as water and/or alcohol together with an essential oil having a selected fragrance or perfume for impregnation into the polymer-liquid composite. These polymer-liquid composite materials are prepared to incorporate interconnected internal pores from about 1 to 500 microns. These polymer-liquid composites often possess oleophilic hydrophobic surfaces and can contain release retarding gums and oils from which hydrophilic and oleophobic vehicles can be expelled by surface forces. Moreover, active ingredients can be incorporated in polymer-liquid composites as soluble particles or precipitates formed by solvent exchange or chemical reaction in situ to provide an internal reservoir which maintains a constant concentration of the active ingredient in solution inside the polymer-liquid composite. Thus, the polymer-liquid composite provides a means for zero-order release in which a nearly uniform level of active ingredient (scent) is maintained throughout the active life of the vehicle. The polymer-liquid composite material provides a means of retarding the evaporative release of volatile materials though diffusive effects and control of effective surface area, as well as through depression in the vapor pressure of the volatile substance. Furthermore, the polymer-liquid composite can be modified by skinning, by coating with a liquid to impede escape of the active ingredient (scent) or by the addition of a release-promoting agent to the environment.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

We claim:

1. A package for an air freshener unit intended primarily for use in the cabin of a motor vehicle and containing a scent emitting material, said package comprising respective front and rear panels shaped to provide there between a first and a second spaced apart pair of compartments for retaining therein respectively an air freshener unit having material that emits a preselected scent and a sample of the material that produces that same scent, said second compartment having an outlet opening communicating therewith and discharging to atmosphere and a finger pressure responsive selected area on said package enabling discharging to atmosphere a puff of the scented air from said second compartment upon applying finger pressure to that selected area.

2. The package as defined in claim 1 wherein said panels each have a truncated conical portion projecting therefrom, wherein said truncated conical projections are disposed in internested relation defining there between said second compartment.

3. The package as defined in claim 1 wherein said second compartment is located in a tab that projects outwardly from the peripheral edge of the package.

4. The package comprising respective front and rear panels so shaped as to provide there between a first and a second spaced apart pair of compartments, an air freshener unit located in said first compartment and having material that emits a preselected scent, a sample of said scent producing material that produces that same scent located in said second compartment, said second compartment having an outlet opening communicating therewith and discharging to atmosphere and a finger pressure responsive selected area on said package enabling discharging to atmosphere a puff of scented air from said second compartment upon applying finger pressure to that selected area.
